(19) United States
(12) Patent Application Publication
(10) Pub. No.: US 2025/0000924 A1
(43) Pub. Date: Jan. 2, 2025

(54) Composition for the prevention, improvement or treatment of allergic disease comprising an extract of Taxus cuspidata as an active ingredient

(71) Applicant: KOREA INSTITUTE OF SCIENCE AND TECHNOLOGY, Seoul (KR)

(72) Inventors: Myung Suk KIM, Gangneung-si (KR); Wook Bin LEE, Gangneung-si (KR); Hye Jin KO, Gangneung-si (KR); Le Thi TAM, Gangneung-si (KR); Tae Kyeom KANG, Gangneung-si (KR); Gun PARK, Gangneung-si (KR)

(73) Assignee: KOREA INSTITUTE OF SCIENCE AND TECHNOLOGY, Seoul (KR)

(21) Appl. No.: 18/748,930

(22) Filed: Jun. 20, 2024

(30) Foreign Application Priority Data
Jun. 20, 2023 (KR) 10-2023-0078630

(51) Int. Cl. A61K 36/13; A61K 45/06; A61P 37/08

(52) U.S. Cl. CPC A61K 36/13; A61K 45/06; A61P 37/08; A61K 2236/333

(57) ABSTRACT

The present invention relates to a composition for the prevention, improvement/amelioration or treatment of allergic disease containing an extract of Taxus cuspidata as an active ingredient, and more specifically, to a composition for the prevention, improvement/amelioration or treatment of allergic respiratory disease comprising an ethanol extract of Taxus cuspidata or a fraction thereof as an active ingredient. In the present invention, the effectiveness of Taxus cuspidata extract in treating allergic respiratory disease was confirmed in an animal model of allergic asthma, and the anti-allergic efficacy of compounds isolated from Taxus cuspidata ethanol extract was confirmed. Therefore, Taxus cuspidata extract of the present invention can be useful as a composition for the prevention, amelioration or treatment of allergic respiratory diseases, particularly allergic asthma or allergic bronchitis.

### CROSS-REFERENCE TO RELATED APPLICATIONS

This application claims the benefit under 35 USC 119(a) of Korean Patent Application No. 10-2023-0078630 filed on Jun. 20, 2023, in the Korean Intellectual Property Office, the entire disclosure of which is incorporated herein by reference for all purposes.

### TECHNICAL FIELD

The present invention relates to a composition for the prevention, improvement/amelioration or treatment of allergic disease containing an extract of Taxus cuspidata as an active ingredient, and more specifically, to a composition for the prevention, improvement/amelioration or treatment of allergic respiratory disease comprising an ethanol extract of Taxus cuspidata or a fraction thereof as an active ingredient.

### BACKGROUND ART

Allergic diseases include anaphylaxis and allergic rhinitis, asthma, atopy, and urticaria, etc. These allergic diseases occur in up to 20% of the population in many countries around the world, and the prevalence is gradually increasing.

Allergic diseases are mostly caused by mediators (mainly histamine, leukotrienes, prostaglandins, TNF-α, cytokines, etc.) released from mast cells in tissues activated by antigen-antibody reactions, and basophils and eosinophils in the blood.

Specifically, allergic asthma is an immune disease of the respiratory mucosa characterized by airway inflammation, mucus hypersecretion, and airway hyperresponsiveness. Airway inflammation is triggered by inflammatory cells such as T cells, B cells, mast cells, neutrophils, and eosinophils, as well as chemical mediators such as cytokines and chemokines, and is characterized by airway hyperresponsiveness, inflammation of the lungs, and increased secretion of IgE in the serum (Herrick C A and Bottomly K., Nat Rev Immunol., 3(5), 405-412, 2003).

Antihistamines, leukodriene inhibitors, and steroids have been used to treat allergic disease, and more recently, protein- and peptide-based histamine and interleukin receptor blockade has been developed to treat allergies. However, the use of allergy medications has been limited to symptomatic relief, and there is an urgent need to develop more fundamental therapeutic drugs (Kips J C, et. al., Am J Respir Crit Care Med., 167(12), 1655-1659, 2003).

Meanwhile, due to a compound called taxol contained in Taxus cuspidata, Taxus cuspidata extract has been shown to have anti-cancer effects against several cancers such as ovarian, lung, and breast cancer. In addition, it has been reported to be effective against influenza, diabetes, hypertension, severe gout, arteriosclerosis, nephritis, and edema (Korean Patent No. 10-1310539), but there are no reports on the anti-allergic effects of Taxus cuspidata extract.

### DISCLOSURE

### Technical Problem

Accordingly, in the present invention, we have made diligent efforts to screen substances that have therapeutic effects on allergic diseases, especially allergic respiratory diseases, and have not only confirmed the therapeutic effect of Taxus cuspidata extract (herein also refer to “extract of Taxus cuspidata”) on allergic respiratory diseases in an animal model of allergic asthma, but also confirmed the anti-allergic efficacy of compounds isolated from Taxus cuspidata ethanol extract (herein also refer to “ethanol extract of Taxus cuspidata”), and completed the present invention.

Accordingly, an object of the present invention is to provide a pharmaceutical composition for the prevention or treatment of allergic respiratory disease comprising Taxus cuspidata extract or a fraction thereof.

Another object of the present invention is to provide a health functional food composition for the prevention or amelioration of allergic respiratory disease comprising Taxus cuspidata extract or fraction thereof.

### Technical Solution

To achieve the above-mentioned purpose, the present invention provides a pharmaceutical composition for preventing or treating allergic respiratory disease containing Taxus cuspidata extract or a fraction thereof as an active ingredient.

In addition, the present invention provides a health functional food composition for preventing or ameliorating allergic respiratory disease containing Taxus cuspidata extract or a fraction thereof as an active ingredient.

In a preferred embodiment of the present invention, the present Taxus cuspidata extract may be extracted using 40% (v/v) to 100% (v/v) ethanol as a solvent.

In another preferred embodiment of the present invention, the present fraction may be a methylene chloride (MC) fraction of 40% (v/v) to 100% (v/v) ethanol extract of Taxus cuspidata.

In another preferred embodiment of the present invention, the allergic respiratory disease may be allergic asthma, allergic bronchitis, or allergic rhinitis.

In another preferred embodiment of the present invention, the present Taxus cuspidata extract or fraction thereof can inhibit the secretion of β-hexosaminidase, an allergen presents in the granules of mast cells, or inhibit the production of prostaglandin E2, TNF-α and interleukin-4 (IL-4) secreted by mast cells.

In another preferred embodiment of the present invention, the present Taxus cuspidata extract or fraction thereof may inhibit or ameliorate allergic symptoms by reducing the number of eosinophils or inflammatory cells in the bronchi; reducing eosinophil infiltration; or reducing IgE in the serum.

In another preferred embodiment of the present invention, the present Taxus cuspidata extract or fraction thereof may comprise one or more compounds selected from the group consisting of:
Taxinine represented by the following formula (1),
Taxinine A represented by the following formula (2),
Taxicatigenin represented by the following formula (5),
4-hydroxyl benzandehyde represented by the following formula (6),
3-(4-Hydroxyphenyl)-1-propanol (3-(4-Hydroxyphenyl)-1-propanol) represented by the following formula (7), and/or
Daucosterol represented by the following formula (9);

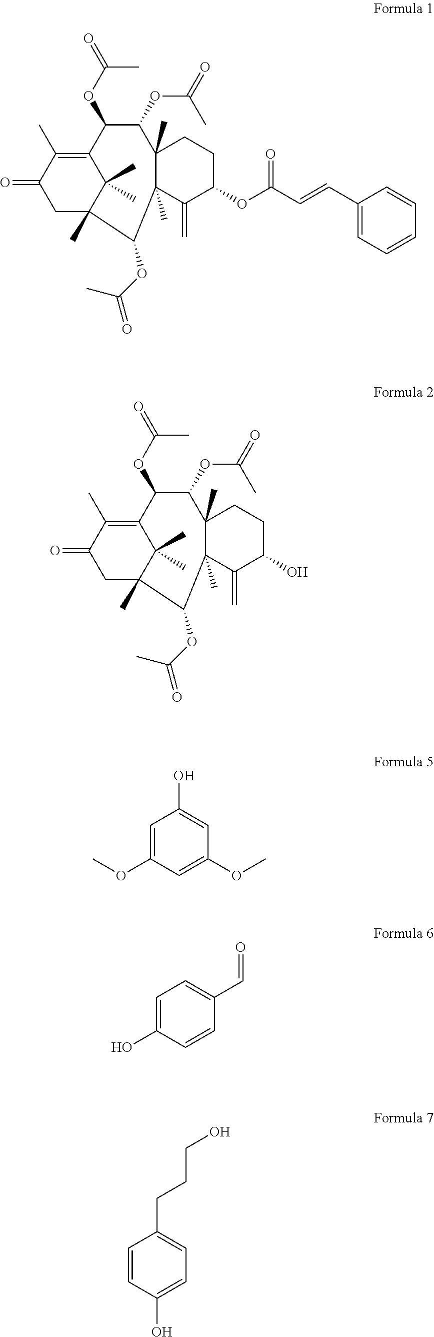

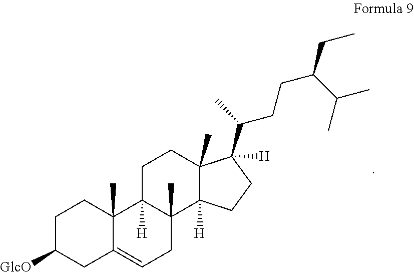

In another preferred embodiment of the present invention, the present Taxus cuspidata extract or fraction thereof may further comprise one or more compounds selected from the group consisting of:
Sciadopitysin represented by the following formula (3),
Ginkgetin represented by the following formula (4), and/or
β-Sitosterol represented by the following formula (8);

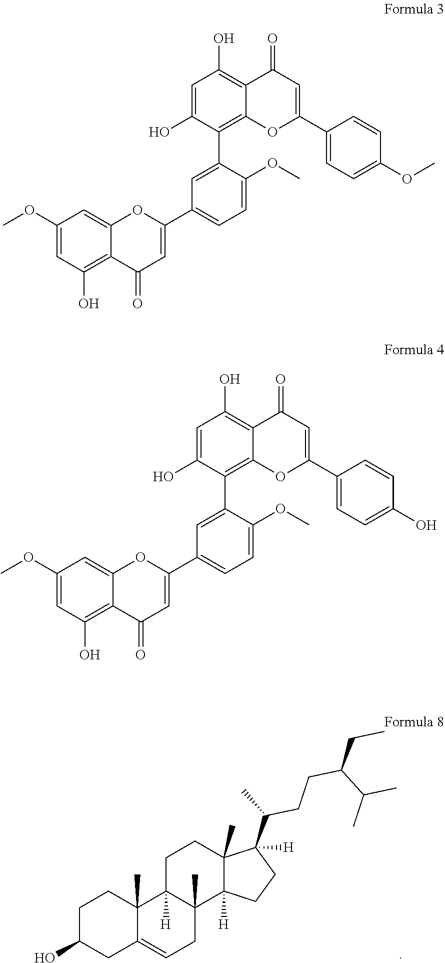

In addition, the present invention provides a method for preventing or treating allergic respiratory disease comprising administering or taking a composition comprising Taxus cuspidata extract or a fraction thereof as an active ingredient to an individual in need.

### Advantageous Effects

In the present invention, the effectiveness of Taxus cuspidata extract in treating allergic respiratory disease was confirmed in an animal model of allergic asthma, and the anti-allergic efficacy of compounds isolated from Taxus cuspidata ethanol extract was confirmed. Therefore, Taxus cuspidata extract of the present invention can be useful as a composition for the prevention, amelioration or treatment of allergic respiratory diseases, particularly allergic asthma or allergic bronchitis.

### DESCRIPTION OF DRAWINGS

FIG. 1 is data confirming the inhibitory effect of the ethanol extract of Taxus cuspidata on allergens in mast cell granules. In FIGS. 1 to 4, Con refers to the control group, IgE refers to the allergy-induced group, TC20 refers to the allergy-inducing group+20 μg/ml of Taxus cuspidata ethanol extract-treated group, and TC40 refers to the allergy-induced group+40 μg/ml of Taxus cuspidata ethanol extract-treated group.

FIG. 2 shows data confirming the inhibitory effect of the ethanol extract of Taxus cuspidata on the production of prostaglandin E2 secreted by mast cells.

FIG. 3 shows data confirming the inhibitory effect of the ethanol extract of Taxus cuspidata on the production of TNF-α secreted by mast cells.

FIG. 4 shows data confirming the inhibitory effect of the ethanol extract of Taxus cuspidata on the production of interleukin-4 secreted by mast cells.

FIG. 5 is a schematic diagram showing a method for producing an allergic asthma animal model.

FIG. 6 shows data confirming the inhibitory effect of Taxus cuspidata ethanol extract on the number of inflammatory cells in bronchoalveolar lavage fluid in an animal model of allergic asthma. In the figures, Monocyte refers to monocyte cells, Lymphocyte refers to lymphocyte cells, and PMN refers to eosinophilic cells. In addition, in FIGS. 6 to 11, Con refers to the normal group, OVA refers to the allergic asthma induced group, DEX refers to the allergic asthma-induced group+dexamethasone-treated group (positive control), TC50 refers to the allergy-inducing group+50 μg/ml of Taxus cuspidata ethanol extract-treated group, TC100 refers to the allergic asthma induction group+100 mg/kg of Taxus cuspidata extract-treated group, and TC200 refers to the allergic asthma induction group+200 mg/kg of Taxus cuspidata extract-treated group.

FIG. 7 shows data confirming the degree of increase in (A) eosinophil cell count and (B) dendritic cell count by Taxus cuspidata ethanol extract through flow cytometry in an allergic asthma animal model.

FIG. 8 is an image of the inhibitory effect of Taxus cuspidata ethanol extract on immune cell infiltration in an animal model of allergic asthma by H&E staining.

FIG. 9 shows (A) images and (B) quantification of the inhibitory effect of Taxus cuspidata ethanol extract on immune cell infiltration by Periodic Acid-Schiff (PAS) staining in an animal model of allergic asthma.

FIG. 10 shows data confirming the suppression of serum IgE and ovalbumin-specific IgE concentrations by Taxus cuspidata ethanol extract in an animal model of allergic asthma.

FIG. 11 shows the analytical data for establishing yield-based optimal extraction and fractionation conditions from Taxus cuspidata ethanol extract.

FIG. 12 is a schematic diagram illustrating a method for isolating compounds from Taxus cuspidata ethanol extract.

FIG. 13 shows data confirming the inhibitory effect of compounds derived from Taxus cuspidata ethanol extract on mast cell degranulation.

FIG. 14 shows data confirming the inhibitory effect of compounds derived from Taxus cuspidata ethanol extract on the production of prostaglandin E2 secreted by mast cells.

FIG. 15 shows data confirming the inhibitory effect of compounds derived from Taxus cuspidata ethanol extract on the production of TNF-α secreted by mast cells.

FIG. 16 shows data confirming the inhibitory effect of compounds derived from Taxus cuspidata ethanol extract on the production of interleukin-4 secreted by mast cells.

### MODE OF THE INVENTION

Hereinafter, the present invention will be described in detail.

The present invention relates to a pharmaceutical composition for preventing or treating allergic respiratory disease containing Taxus cuspidata as an active ingredient.

In addition, the present invention relates to a functional health food composition for preventing or ameliorating allergic respiratory disease containing Taxus cuspidata as an active ingredient.

In the present invention, Taxus cuspidata extract can be extracted using ethanol, preferably 40% (v/v) to 100% (v/v) ethanol, more preferably 45% (v/v) to 95% (v/v) ethanol, as a solvent. The present Taxus cuspidata extract can be prepared using conventional solvents according to methods known in the art, but solvents other than 40% (v/v) to 100% (v/v) ethanol may result in a lower content or no content of the substance with anti-allergic activity in the Taxus cuspidata extract.

In the present invention, the fraction can be a methylene chloride (MC) fraction of 40% (v/v) to 100% (v/v) ethanol extract of Taxus cuspidata.

In the present invention, the allergic respiratory disease can be allergic asthma, allergic bronchitis, or allergic rhinitis.

In the present invention, Taxus cuspidata extract or fraction thereof can inhibit the secretion of β-hexosaminidase, an allergen presents in the granules of mast cells, or inhibit the production of prostaglandin E2, TNF-α and interleukin-4 (IL-4) secreted by mast cells.

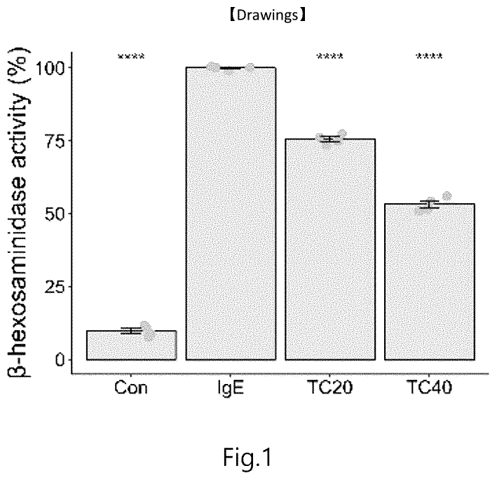

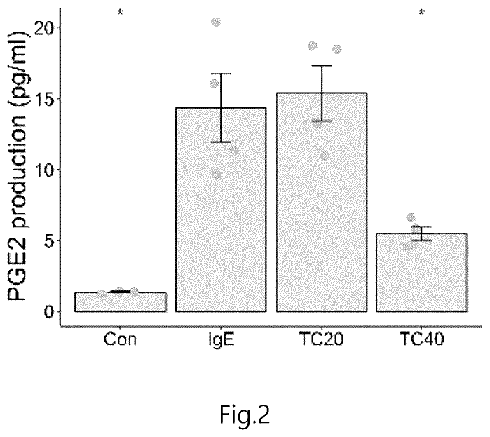

In a specific embodiment of the present invention, a 50% ethanol extract of Taxus cuspidata was prepared and its anti-allergic effect was confirmed. As a result, it was confirmed that the secretion of allergens present in mast cell granules was inhibited by the Taxus cuspidata ethanol extract (FIG. 1), and the production of prostaglandin E2, TNF-α and interleukin-4 (IL-4) secreted by mast cells was inhibited (FIGS. 2 to 4).

In the present invention, Taxus cuspidata extract or fraction thereof can inhibit or ameliorate allergic symptoms by reducing the number of eosinophils or inflammatory cells in the bronchi; reducing eosinophil infiltration; or reducing IgE in the serum.

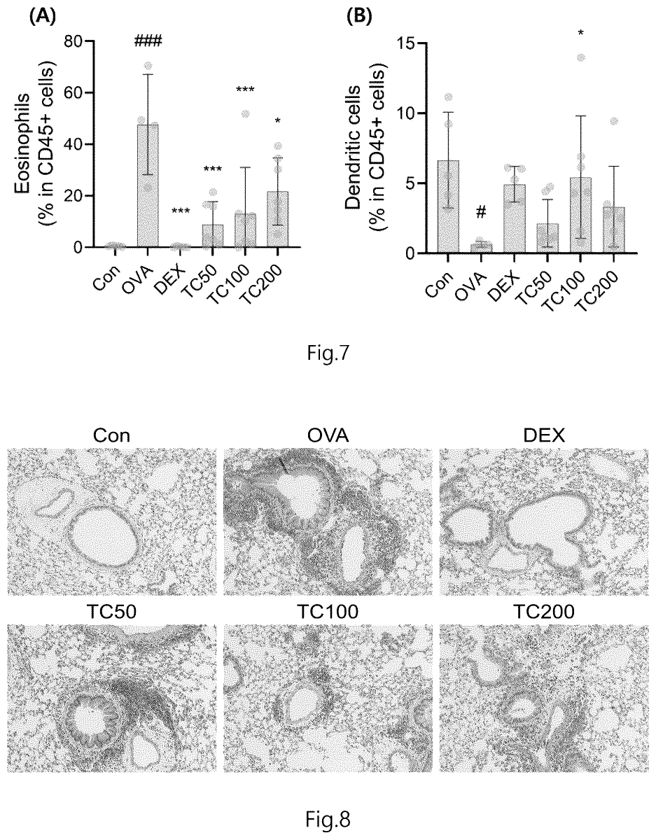

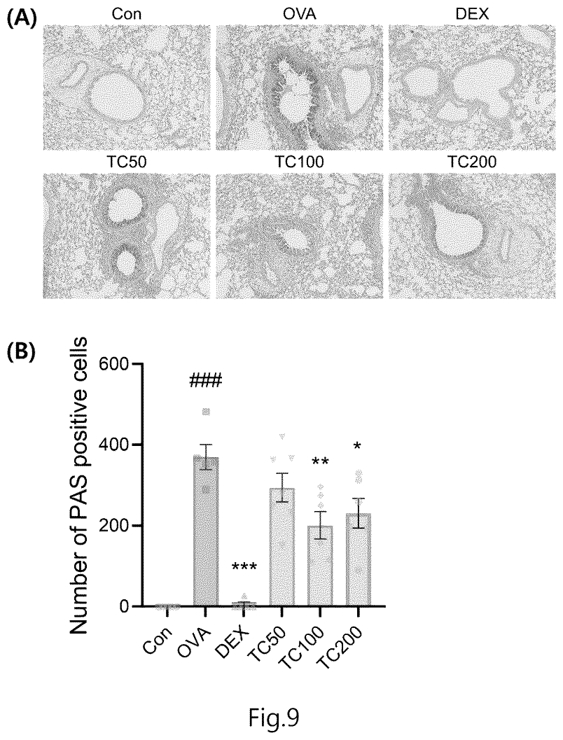

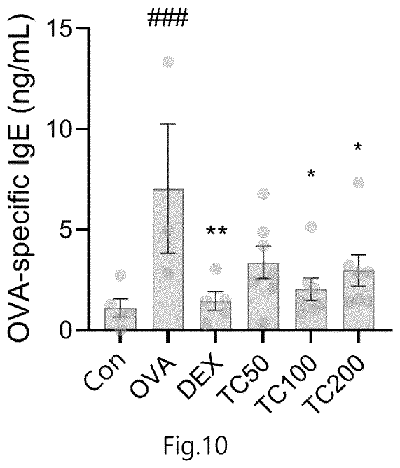

In another specific embodiment of the present invention, an animal model for inducing bronchial asthma, an allergic respiratory disease, was produced and treated with ethanol extract of Taxus cuspidata. As a result, it was confirmed that in the ethanol extract-treated group, the number of neutrophils, eosinophils, and macrophages in the bronchoalveolar lavage fluid decreased and the number of dendritic cells increased (FIGS. 6 and 7), infiltration of eosinophils, which are immune cells, was suppressed (FIGS. 8 and 9), and the concentrations of IgE and ovalbumin-specific IgE in serum, which correlated with the severity of asthma, were suppressed (FIG. 10).

In the present invention, Taxus cuspidata extract or fraction thereof can comprise one or more compounds selected from the group consisting of:
Taxinine represented by the following formula (1),
Taxinine A, represented by the following formula (2),
Taxicatigenin represented by the following formula (5),
4-hydroxyl benzandehyde represented by the following formula (6),
3-(4-Hydroxyphenyl)-1-propanol (3-(4-Hydroxyphenyl)-1-propanol) represented by the following formula (7), and/or
Daucosterol represented by the following formula (9);

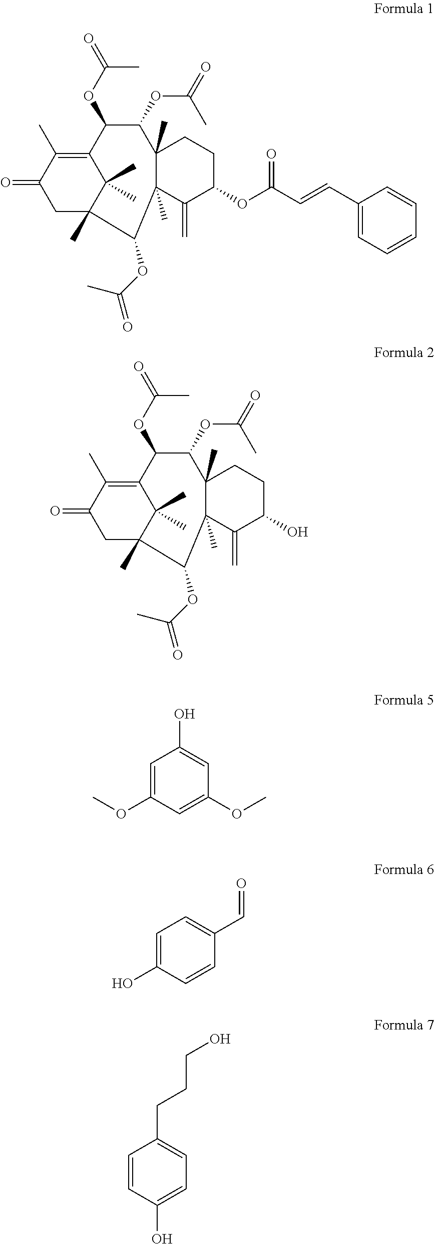

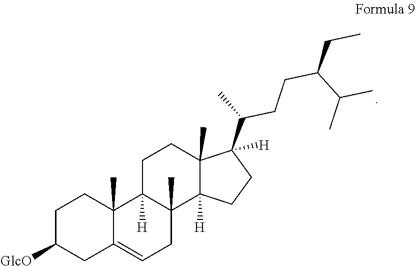

In the present invention, Taxus cuspidata extract or fraction thereof can further comprise one or more compounds selected from the group consisting of:
Sciadopitysin represented by the following formula (3),
Ginkgetin represented by the following formula (4), and/or
β-Sitosterol represented by the following formula (8);

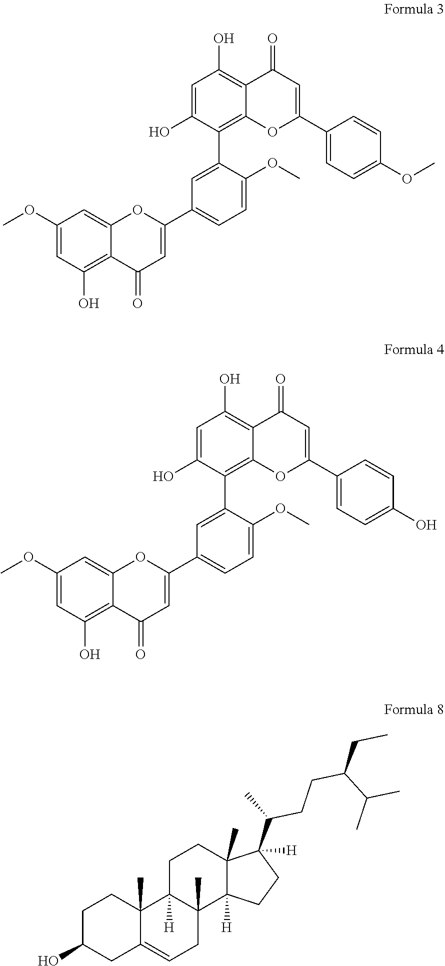

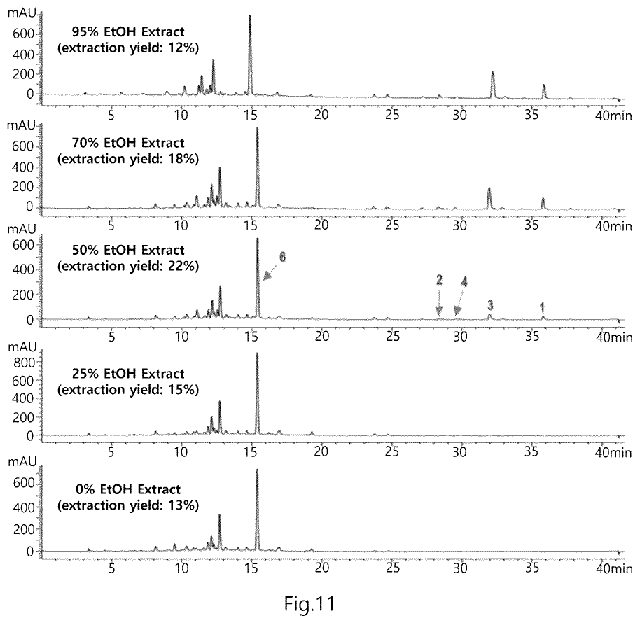

In another specific embodiment of the present invention, optimal extraction and fractionation conditions were established from 0%, 25%, 50%, 70%, or 95% ethanol extracts of Taxus cuspidata, and it was found that the 50%, 70%, and 95% ethanol extracts differed from the 0% and 25% ethanol extracts in the types of active constituents that were isolated (FIG. 11).

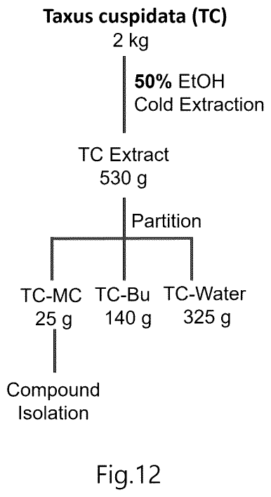

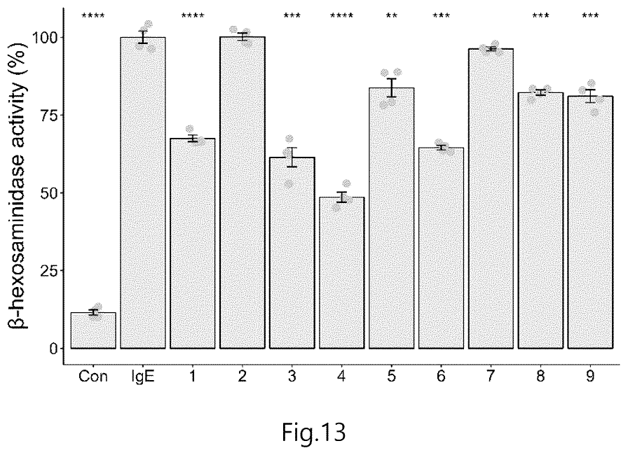

Furthermore, 9 compounds isolated from the methylene chloride fraction of the 50% ethanol extract of Taxus cuspidata using the method shown in the schematic diagram of FIG. 12 inhibit the degranulation of mast cells and increase the production of prostaglandin E2, TNF-α, and interleukin-4. Inhibition was confirmed (FIGS. 13 to 16). In particular, it was confirmed that the anti-allergy effects of Taxinine A, Taxicatigenin, 4-hydroxyl benzandehyde and 3-(4-Hydroxyphenyl)-1-propanol compounds were excellent.

The pharmaceutical compositions of the present invention can be formulated and used in various forms, each according to methods known in the art. For example, they can be formulated into oral dosage forms, such as pills, granules, tablets, capsules, suspensions, emulsions, syrups, and the like, and can be formulated into topical preparations, suppositories, and sterile injectable solutions. Each formulation may further comprise pharmaceutically acceptable carriers, excipients and diluents. It can also be formulated and used in the form of topical agents and sterile injectable solutions, such as pills, granules, tablets, capsules, suspensions, emulsions, syrups, aerosols, and the like, in accordance with the methods of the art.

The carriers, excipients and diluents include lactose, dextrose, sucrose, oligosaccharides, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, mineral oil, and the like. The pharmaceutical compositions are prepared or formulated using commonly used diluents or excipients such as fillers, extenders, binders, wetting agents, disintegrants, surfactants, and surfactants.

Solid dosage forms for oral administration include tablets, pills, powders, granules, capsules, and the like, which are prepared by mixing the composition with at least one excipient, such as starch, calcium carbonate, sucrose, lactose, gelatin, and the like. In addition to simple excipients, lubricants such as magnesium stearate talc are also used. Liquid preparations for oral administration include suspensions, solutions, emulsions, and syrups, which may contain a variety of excipients, such as wetting agents, sweeteners, flavors, and preservatives, in addition to the commonly used simple diluents of water and liquid paraffin. Formulations for parenteral administration include sterile aqueous solutions, non-aqueous solutions, suspensions, emulsions, lyophilized preparations, and suppositories. Non-aqueous solvents and suspensions may include propylene glycol, polyethylene glycol, vegetable oils such as olive oil, and injectable esters such as ethylolate. As a base for suppositories, witepsol, macrogol, tween 61, cacao paper, laurin paper, glycerceratin, etc. can be used.

As used in the present invention, the term “administering” refers to providing an individual with a pharmaceutical composition of the invention by any suitable method. The pharmaceutical compositions of the present invention can be administered in a therapeutically effective amount, which is an amount of the active ingredient or pharmaceutical composition that induces a biological or medical response in a tissue system, animal, or human as conceived by the researcher, veterinarian, physician, or other clinician, i.e., an amount that induces relief of the symptoms of the disease or disorder being treated. It will be apparent to those skilled in the art that the therapeutically effective dosage and frequency of administration of the pharmaceutical compositions of the present invention will vary depending on the desired effect. Therefore, the optimal dosage to be administered is readily determined by those skilled in the art and may be adjusted according to a variety of factors, including the type of disease, the severity of the disease, the content of the active ingredient and other ingredients in the composition, the type of formulation, the age, weight, general health, gender and diet of the patient, the time of administration, the route of administration and the distribution of the composition, the duration of treatment, and concomitant medications. The pharmaceutical compositions of the present invention can be administered to an individual by a variety of routes. For example, but not limited to, intravenous, intraperitoneal, intramuscular, intra-arterial, oral, intracardiac, intramedullary, intrathecal, transdermal, transplant, enteral, subcutaneous, sublingual, or topical administration. The pharmaceutical compositions of the present invention can be administered in an amount from 1 to 10,000 mg/kg/day, and can be administered once daily or in multiple doses.

The health functional food composition of the present invention can be used as a health functional food, food additive, or dietary supplement. When using the composition of the present invention as a food additive, it can be used appropriately according to conventional methods, such as adding it as is or mixing it with other foods or food ingredients.

Additionally, the mixing amount of the health functional food composition may be appropriately changed depending on the purpose of use (prevention, health, or therapeutic treatment). As a specific example, when producing a food or beverage, the composition of the present invention can be added in an amount of 15% by weight or less, preferably 10% by weight or less, based on the raw materials. However, when consumed for a long period of time for health and hygiene purposes or health control, it can be added in an amount below the above range. Since there is no problem in terms of safety, the active ingredient can be used in an amount above the above range.

There is no particular limitation on the type of food, but examples of food to which the composition of the present invention can be added include meat, sausages, bread, chocolate, candy, snacks, confectionery, pizza, ramen, other noodles, gum, and ice cream. It includes dairy products, various soups, beverages, tea, drinks, alcoholic beverages, vitamin complexes, etc., and includes all health foods in the conventional sense.

When the health functional food composition of the present invention can be formulated as beverages, they may contain additional ingredients such as various flavoring agents or natural carbohydrates as in conventional beverages. The natural carbohydrates may include monosaccharides such as glucose and fructose; disaccharides such as maltose and sucrose; natural sweeteners such as dextrin and cyclodextrin; and synthetic sweeteners such as saccharin and aspartame. The natural carbohydrates may comprise from 0.01 to 10 wt %, preferably from 0.01 to 0.1 wt %, based on the total weight of the food composition of the invention.

The health functional food composition of the present invention can contain various nutrients, vitamins, electrolytes, flavors, colorants, pectic acid and its salts, alginic acid and its salts, organic acids, protective colloidal thickeners, pH adjusters, stabilizers, preservatives, glycerin, and alcohol., carbonating agents used in carbonated beverages, etc., and may include pulp for the production of natural fruit juice, fruit juice drinks, and vegetable drinks, but are not limited thereto. These ingredients can be used independently or in combination. The ratio of the above additives is not greatly limited, but is preferably contained within the range of 0.01 to 0.1% by weight based on the total weight of the food composition of the present invention.

Hereinafter, the present invention will be described in more detail through examples.

These examples are only for illustrating the present invention, and it will be obvious to those skilled in the art that the scope of the present invention should not be construed as limited by these examples.

### Example 1

Preparation of Taxus cuspidata Extract

Taxus cuspidata was purchased from Herb Village Co., Ltd), and cut into appropriate sizes. 2 kg of the cut Taxus cuspidata and 24 L of 50% ethanol were added to the extraction vessel and subjected to cold extraction, and then filtered through extraction solvent filter paper. The extraction process was repeated twice, and then the solvent was concentrated and dried under reduced pressure to obtain 530 g of the present extract.

### Example 2

Inhibitory Effect of Taxus cuspidata Extract on Secretion of Allergens within Mast Cell Granules

In the present invention, it was confirmed whether Taxus cuspidata extract inhibits the secretion of allergens present in the granules of mast cells.

First, rat basophilic leukocytes (RBL-2H3, American Type Culture Collection, USA) mast cells were cultured in minimal medium in the presence of antibiotics and 10% serum. After incubation, the cells were harvested with trypsin and seeded in 24-well microtiter plates at 2×10⁵ cells/well and cultured until 80% confluent. To these cells, the medium was replaced with PIPES buffer (25 mM PIPES, pH 7.2, 159 mM NaCl, 5 mM KCl, 0.4 mM MgCl₂, 1 mM CaCl₂, 5.6 mM glucose, and 0.1% BSA), and then the Taxus cuspidata ethanol extract prepared in Example 1 above was added to concentrations of 20 μg/ml and 40 μg/ml, respectively, and incubated for 1 hour. After 1 hour, DNP-BSA was added to a final concentration of 200 μg/ml and stimulation was induced for 30 minutes.

The degree of secretion of allergens was determined by measuring the activity of β-hexosaminidase, a marker of degranulation in the medium, and the activity of β-hexosaminidase was determined as the amount of p-nitrophenol liberated from p-nitrophenyl-acetyl-β-D-glucosaminide (Funaba M. et al, Cell Biol. Int., 27:879-85, 2003).

As a result, it was confirmed that the ethanol extract of Taxus cuspidata effectively inhibited the secretion of allergens present in the granules of mast cells in a concentration-dependent manner, as shown in FIG. 1.

### Example 3

Inhibitory Effect of Taxus cuspidata Extract on Prostaglandin E2 Production Secreted by Mast Cells

In the present invention, it was confirmed whether Taxus cuspidata extract inhibits the production of prostaglandin E2 (PGE2) in mast cells.

First, RBL-2H3 mast cells were seeded at 2×10⁵ cells/well in a 24-well microtiter plate containing Dulbecco's Modified Eagle's Media (DMEM) medium containing 2.5% fetal bovine serum, and cultured until 80% confluence. Then, the ethanol extract of Taxus cuspidata prepared in Example 1 above was added to the serum-free DMEM medium to a concentration of 20 μg/ml and 40 μg/ml, respectively, and the mast cells were incubated for 24 hours. After the mast cells were treated with DNP-BSA for 30 min, the cell culture medium was collected and centrifuged, and only the supernatant was harvested. The amount of prostaglandin E2 in the supernatant was determined using an ELISA kit (R&D systems, USA).

As a result, as shown in FIG. 2, it was confirmed that the ethanol extract of Taxus cuspidata effectively inhibited the production of prostaglandin E2 in mast cells in a concentration-dependent manner.

### Example 4

Inhibitory Effect of Taxus cuspidata Extract on TNF-α Production Secreted by Mast Cells

In the present invention, it was confirmed whether Taxus cuspidata extract inhibits the production of TNF-α in mast cells.

First, RBL-2H3 mast cells were seeded at 2×10⁵ cells/well in a 24-well microtiter plate containing Dulbecco's Modified Eagle's Media (DMEM) medium containing 2.5% fetal bovine serum, and cultured until 80% confluence. Then, the ethanol extract of Taxus cuspidata prepared in Example 1 above was added to the serum-free DMEM medium to a concentration of 20 μg/ml and 40 μg/ml, respectively, and the mast cells were incubated for 24 hours. After the mast cells were treated with DNP-BSA for 30 min, the cell culture medium was collected and centrifuged, and only the supernatant was harvested. The amount of TNF-α in the supernatant was determined using an ELISA kit (Invitrogen, USA).

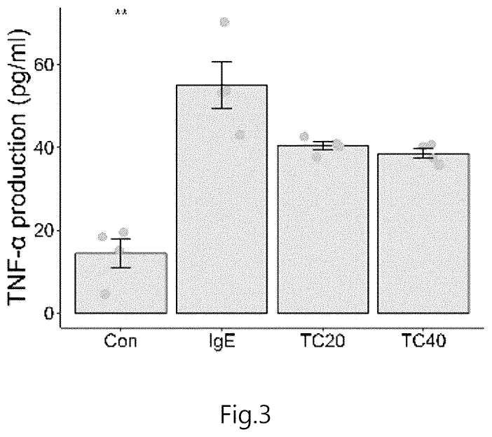

As a result, as shown in FIG. 3, it was confirmed that the ethanol extract of Taxus cuspidata effectively inhibited TNF-α production in mast cells in a concentration-dependent manner.

### Example 5

Inhibitory Effect of Taxus cuspidata Extract on Interleukin-4 Production Secreted by Mast Cells

In the present invention, it was confirmed whether Taxus cuspidata extract inhibits the production of interleukin-4 (IL-4) in mast cells.

First, RBL-2H3 mast cells were seeded at 2×10⁵ cells/well in a 24-well microtiter plate containing Dulbecco's Modified Eagle's Media (DMEM) medium containing 2.5% fetal bovine serum, and cultured until 80% confluence. Then, the ethanol extract of Taxus cuspidata prepared in Example 1 above was added to the serum-free DMEM medium to a concentration of 20 μg/ml and 40 μg/ml, respectively, and the mast cells were incubated for 24 hours. After the mast cells were treated with DNP-BSA for 30 minutes, the cell culture medium was collected and centrifuged, and only the supernatant was harvested. The amount of interleukin-4 in the supernatant was determined using an ELISA kit (Invitrogen, USA).

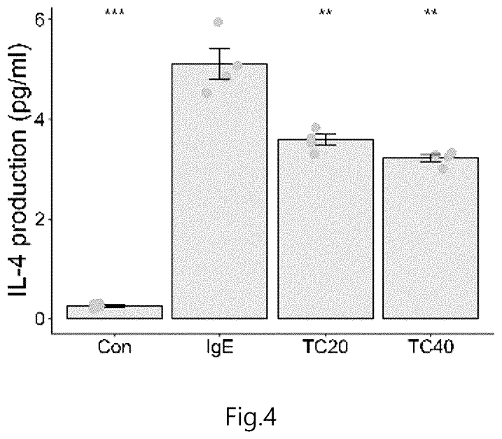

As a result, as shown in FIG. 4, it was confirmed that the ethanol extract of Taxus cuspidata effectively inhibited the production of interleukin-4 in mast cells in a concentration-dependent manner.

### Example 6

Production of Animal Model for Inducing Allergic Bronchial Asthma and Administration of Taxus cuspidata Extract

To create the allergic bronchial asthma induction model, 60 6-week-old Balb/c female mice with an average body weight of approximately 20 g were purchased from OrientBio, and the animals were acclimatized in the laboratory environment (25±1° C. temperature and 40±5% humidity) for 1 week, and individuals with no abnormalities observed on basic physical examination were selected.

Mice were divided into six groups: normal (Control, PBS), allergic asthma-induced (OVA), OVA+dexamethasone (Dexamethasone; Sigma Aldrich), and OVA+three concentrations of the ethanol extract of Taxus cuspidata (TC, 50 mg/kg, 100 mg/kg, 200 mg/kg). Dexamethasone, a steroid drug used to treat bronchial asthma, was used as a positive control.

Allergic bronchial asthma was induced by sensitization with ovalbumin (OVA; grade V; Sigma-Aldrich, USA) and aluminum hydroxide (Al(OH)₃, Sigma Aldrich), followed by inhalation of OVA using an ultrasonic nebulizer.

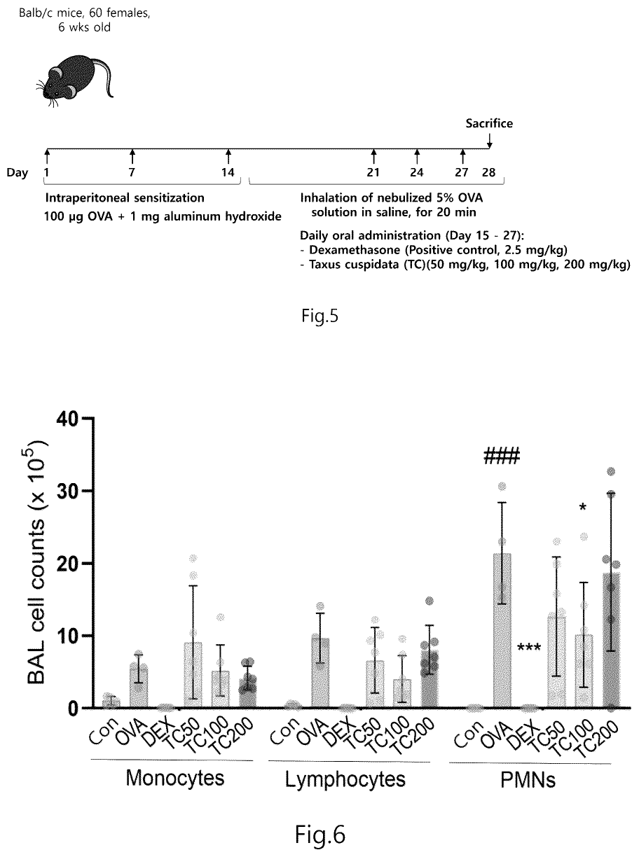

Specifically, as shown in the schematic in FIG. 5, six groups of mice were injected intraperitoneally with 100 μg OVA and 1 mg Al(OH)₃ on days 1, 7, and 14, and then sensitized mice were inhaled with 5% OVA for 20 minutes on days 21, 24, and 27 using an ultrasonic nebulizer. For the dexamethasone-treated group, dexamethasone was dissolved in a 0.5% (v/v) carboxymethylcellulose (CMC) solution and administered orally at 2.5 mg/kg on days 15 through 27. The three concentrations of Taxus cuspidata ethanol extract-treated groups were orally administered at 50, 100, and 200 mg/kg of the ethanol extract of Taxus cuspidata dissolved in 0.5% (v/v) CMC solution with OVA inhalation from day 15 to day 27, and mice were sacrificed on day 28.

### Example 7

Inhibitory Effect of Taxus cuspidata Extract on the Number of Inflammatory Cells in Bronchoalveolar Lavage Fluid

To determine the anti-allergic asthma activity of the samples obtained in Example 7 above, the following experiments were performed by applying the method for evaluating inflammatory cells in bronchoalveolar lavage fluid described in the literature (Chang, Y. et al., Allergy Asthma Immunol Res, 14:99-116, 2022).

To obtain bronchoalveolar lavage fluid, an 18G catheter was inserted into the bronchus and bronchoalveolar lavage fluid was collected from the lungs by gently flushing the bronchus three times with 1 ml of sterile PBS containing 2% fetal bovine serum (FBS).

To determine the number of inflammatory cells in bronchoalveolar lavage fluid, each mouse bronchoalveolar lavage fluid collected was stained with trypan blue (T8154, Sigma) and the total number of cells, excluding dead cells, was counted using a hemocytometer (MIS-1401, Superior). The specimens were then smeared using a centrifuge for cell collection (Cytospin, Shandon™), followed by Diff-Quick staining (Sysmex, Switzerland) to differentially count eosinophils and other inflammatory cells.

As a result, as shown in FIG. 6, the number of eosinophils and total inflammatory cells was significantly increased in the asthma-induced group (OVA) compared to the normal control group (Con), and the increased total inflammatory cell count and eosinophil count were significantly decreased in both the dexamethasone-treated and Taxus cuspidata extract-treated groups (TC).

### Example 8

Inhibitory Effect of Taxus cuspidata Extracts on the Number of Inflammatory Cells in Bronchoalveolar Lavage Fluid by Flow Cytometry Analysis

Immunofluorescence staining was performed at 4° C. on cells in bronchoalveolar lavage fluid obtained in Example 7 above.

Anti-CD45-APC, anti-CD11c-FITC, and anti-F4/80-PE were used to stain eosinophils and dendritic cells. After reacting at 4° C. for 1 hour, washed three times with phosphate-buffered saline, inflammatory cells were analyzed by flow cytometry (BD FACSVerse™ Cell Analyzer, BD Biosciences) (FIG. 7A and FIG. 7B).

As a result, as shown in FIG. 7A, the number of eosinophils was significantly increased in the asthma-induced group (OVA) compared to the normal control group (Con), and the increased eosinophil count was significantly decreased in both the dexamethasone and Taxus cuspidata extract treatment groups.

Furthermore, as shown in FIG. 7B, dendritic cells were significantly reduced in the asthma-induced group (OVA) compared to the normal control group (Con), and the reduced dendritic cell numbers were significantly reduced in both the dexamethasone-treated and Taxus cuspidata extract-treated groups.

### Example 9

Inhibitory Effect of Immune Cell Infiltration of Taxus cuspidata Extract Through Histopathological Analysis

To confirm the infiltration of inflammatory cells, lungs were harvested from each group of mice in Example 6 above, and then subjected to conventional formalin fixation and paraffin embedding, followed by 4 um-thick cryosections for permatoxylin and eosin (H&E) staining.

As a result, as shown in FIG. 8, unlike the normal group, in the allergic asthma induction group by OVA treatment, eosinophils infiltrated through the blood vessels of the lungs and the number of eosinophils was significantly increased, but the infiltration of eosinophils was significantly inhibited in the dexamethasone treatment group and Taxus cuspidata extract treatment group.

Periodic Acid-Schiff (PAS) staining was also performed to assess hyperplasia of lung tissue goblet cells, which are responsible for mucus and secretion. The PAS-stained goblet cells were observed by light microscopy along the lung vessels, and as shown in FIG. 9, the number of goblet cells was significantly increased in the allergic asthma induction group, while the infiltration of eosinophils was significantly inhibited in the dexamethasone and Taxus cuspidata extract treatment groups.

### Example 10

Inhibitory Effect of Taxus cuspidata Extract on Serum IgE and Ovalbumin-Specific IgE Concentration

Immunoenzymatic assays were used in serum to measure IgE and ovalbumin-specific IgE (OVA-specific IgE), which correlate with asthma severity.

Serum from each group in Example 7 above was reacted in an ELISA kit (BioLegend) for the determination of OVA-specific IgE concentration. After stopping the reaction with 2N sulfuric acid solution, the spectral absorbance was measured using an absorbance meter at 450 nm.

As a result, as shown in FIG. 10, the concentration of ovalbumin-specific IgE in serum was significantly increased in the asthma induction group (OVA), while the concentration of ovalbumin-specific IgE antibodies was significantly decreased in the dexamethasone and Taxus cuspidata extract treatment groups.

Thus, it was confirmed that the present Taxus cuspidata extract can be useful in the prevention or treatment of allergies and asthma by inhibiting the concentration of ovalbumin-specific IgE.

### Example 11

Isolation of Active Ingredients Derived from Taxus cuspidata Extract

In the present invention, yield-based optimal extraction and fractionation conditions were established from Taxus cuspidata extract to isolate the active ingredients with anti-allergic effect from Taxus cuspidata extract.

First, 2 kg of appropriately sized Taxus cuspidata was cold-soxhleted with a mixture of 0% (100% water), 25%, 50%, 70%, or 95% ethanol, respectively, and then filtered through extraction solvent filter paper to obtain the extract. The Taxus cuspidata extracts prepared above were analyzed for chemical composition by HPLC, LC-MS, and NMR. The extracts were loaded at 10 mg/ml and subjected to HPLC analysis. Table 1 below shows the HPLC analysis conditions, and Table 2 shows the compound names corresponding to each peak number in FIG. 11. The extracts were then subjected to gradient fractionation with methylene chloride (MC), hexane and water.

As shown in FIG. 11, the extraction yields of the 50% ethanol extract and the 70% ethanol extract were higher, and in particular, the 50%, 70%, and 95% ethanol extracts were found to have different types of active ingredients separated from the 0% and 25% ethanol extracts.

Accordingly, the present inventors utilized a 50% ethanol extract with a high extraction yield to isolate nine active components from the methylene chloride fraction of the 50% ethanol extract of Taxus cuspidata, as shown in the schematic diagram of FIG. 12 (Table 2). Specifically, 2 kg of Taxus cuspidata was extracted twice with 50% ethanol, and the solvent was subsequently concentrated under reduced pressure and dried to yield 530 g of extract. The methylene chloride layer was then further separated using a silica gel open column under a hexane-ethyl acetate gradient (10:0 to 0:10) to obtain six fractions. The first, third, and fifth isolates of the six fractions were separated by molecular weight using a Sephadex open column under 50% methanol, and nine compounds were obtained by prep-MPLC.

TABLE 1
HPLC analysis conditions

| | Water | Acetonitrile |
|---|---|---|
| Time | (0.1% formic acid) | (0.1% formic acid) |
| 0 | 95 | 5 |
| 5 | 90 | 10 |
| 15 | 80 | 20 |
| 30 | 70 | 30 |
| 35 | 50 | 50 |
| 42 | 0 | 100 |
| 50 | 5 | 95 |

Column: Hydrosphere C18 (Ømm, 5 μm)
Flow rate: 1.0 mL/min
Injection volume: 10 μl
Detection wavelength: 280 nm
Running time: 50 min
Column temperature: 30° C.

TABLE 2
9 compounds derived from Taxus cuspidata extract

| No. | Compound | |
|---|---|---|
| 1 | Taxinin | Formula 1 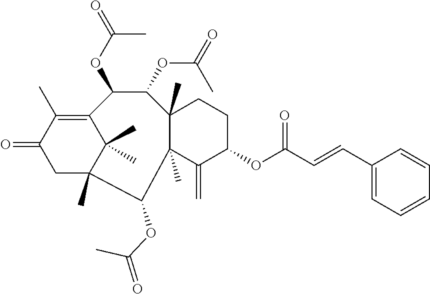 |
| 2 | Taxinin A | Formula 2 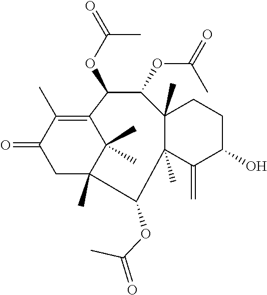 |
| 3 | Sciadopitysin | Formula 3 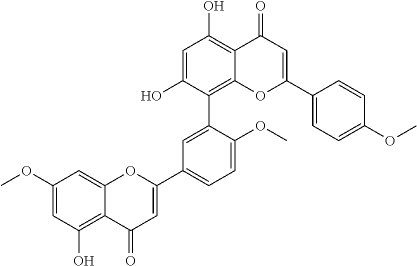 |
| 4 | Ginkgetin | Formula 4 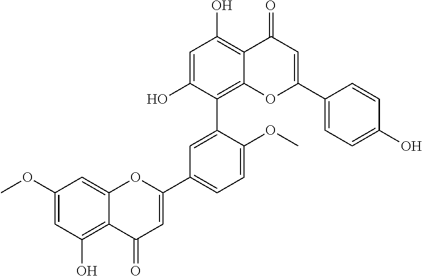 [SMILES: COc1cc(O)c2c(=O)cc(-c3ccc(OC)c(-c4c(O)cc(O)c5c(=O)cc(-c6ccc(O)cc6)oc45)c3)oc2c1] |
| 5 | Taxicatigenin | Formula 5 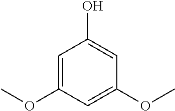 |
| 6 | 4-hydroxyl benzandehyde | Formula 6 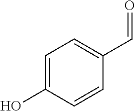 |
| 7 | 3-(4-Hydroxyphenyl)-1-propanol | Formula 7 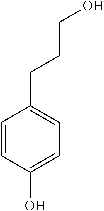 [SMILES: OCCCc1ccc(O)cc1] |
| 8 | β-sitosterol | Formula 8 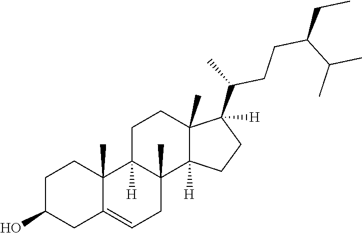 |
| 9 | Daucosterol | Formula 9 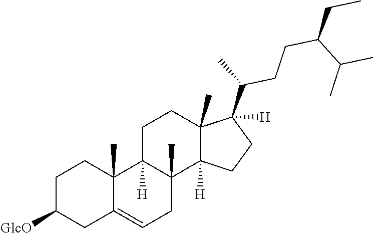 |

### Example 12

Anti-Allergic Effect of Active Ingredients Derived from Taxus cuspidata Extract

### 12-1: Mast Cell Degranulation Inhibition Effect

Mast cells were cultured in the same manner as in Example 2 above, and then treated with the nine compounds isolated in Example 11 above at a concentration of 20 μM, respectively.

As a result, as shown in FIG. 13, it was confirmed that mast cell degranulation was inhibited by 9 compounds derived from the ethanol extract of Taxus cuspidata.

### 12-2: Inhibitory Effect on Prostaglandin E2 Production

Mast cells were cultured in the same manner as in Example 3 above, and then treated with the nine compounds isolated in Example 11 above at a concentration of 20 μM, respectively.

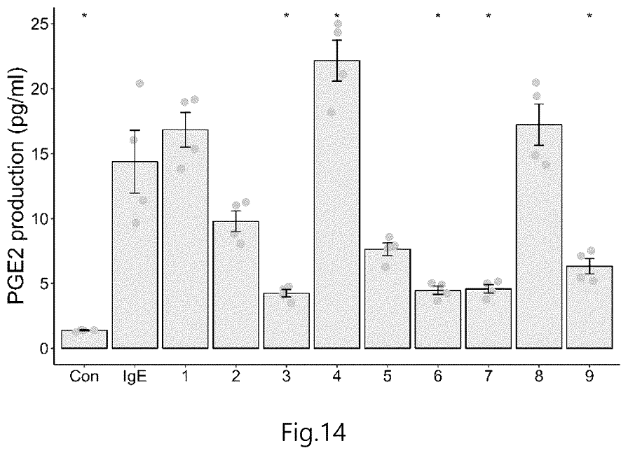

As a result, as shown in FIG. 14, it was confirmed that the production of prostaglandin E2 secreted by mast cells was inhibited by 9 types of compounds derived from the ethanol extract of Taxus cuspidata.

### 12-3: Inhibitory Effect on TNF-α Production

treated with the 9 compounds isolated in Example 11 at a concentration of 20 μM, respectively.

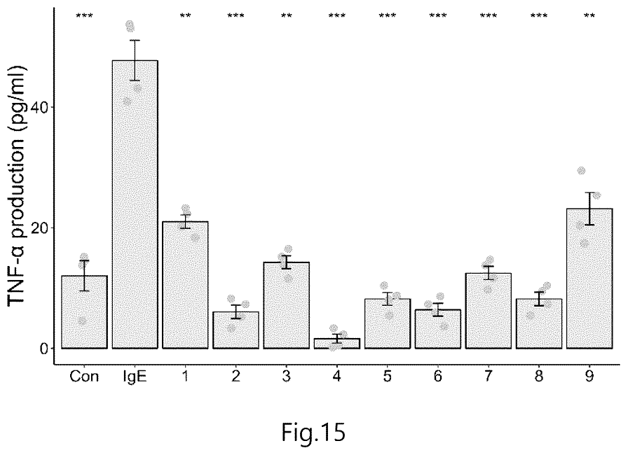

As a result, as shown in FIG. 15, it was confirmed that the production of TNF-α secreted by mast cells was suppressed by 9 compounds derived from the ethanol extract of Taxus cuspidata.

### 12-4: Interleukin-4 Production Inhibition Effect

Mast cells were cultured in the same manner as in Example 5 above, and then treated with the nine compounds isolated in Example 11 above at a concentration of 20 μM, respectively.

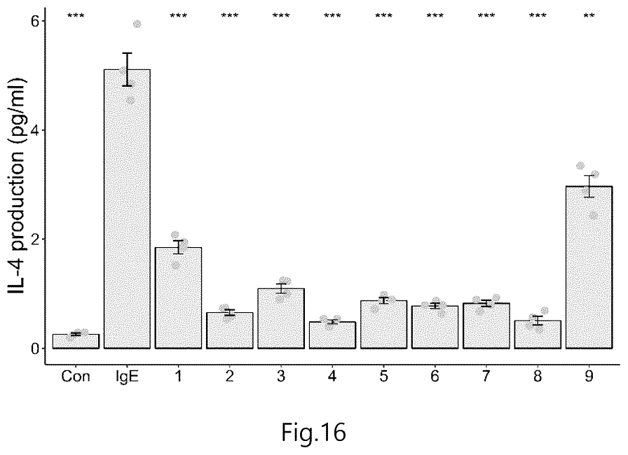

As a result, as shown in FIG. 16, it was confirmed that the production of interleukin-4 secreted by mast cells was suppressed by 9 compounds derived from the ethanol extract of Taxus cuspidata.

1. A pharmaceutical composition for preventing or treating allergic respiratory disease, comprising Taxus cuspidata extract or a fraction thereof as an active ingredient.

2. The pharmaceutical composition for preventing or treating allergic respiratory disease of claim 1, wherein Taxus cuspidata extract is extracted using 40% (v/v) to 100% (v/v) ethanol as a solvent.

3. The pharmaceutical composition for preventing or treating allergic respiratory disease of claim 1, wherein the fraction is a methylene chloride (MC) fraction of a 40% (v/v) to 100% (v/v) ethanol extract of Taxus cuspidata.

4. The pharmaceutical composition for preventing or treating allergic respiratory disease of claim 1, wherein the allergic respiratory disease is allergic asthma, allergic bronchitis, or allergic rhinitis.

5. The pharmaceutical composition for preventing or treating allergic respiratory disease of claim 1, wherein Taxus cuspidata extract or fraction thereof inhibits the secretion of β-hexosaminidase, an allergen presents in the granules of mast cells, or inhibits the production of prostaglandin E2, TNF-α and interleukin-4 (IL-4) secreted by mast cells.

6. The pharmaceutical composition for preventing or treating allergic respiratory disease of claim 1, wherein Taxus cuspidata extract or fraction thereof inhibits or ameliorates allergic symptoms by reducing the number of eosinophils or inflammatory cells in the bronchi; reducing eosinophil infiltration; or reducing IgE in the serum.

7. The pharmaceutical composition for preventing or treating allergic respiratory disease of claim 1, wherein Taxus cuspidata extract or fraction thereof comprises one or more compounds selected from the group consisting of:
Taxinine represented by the following formula (1),
Taxinine A represented by the following formula (2),
Taxicatigenin represented by the following formula (5),
4-hydroxyl benzandehyde represented by the following formula (6),
3-(4-Hydroxyphenyl)-1-propanol (3-(4-Hydroxyphenyl)-1-propanol) represented by the following formula (7), and/or
Daucosterol represented by the following formula (9);

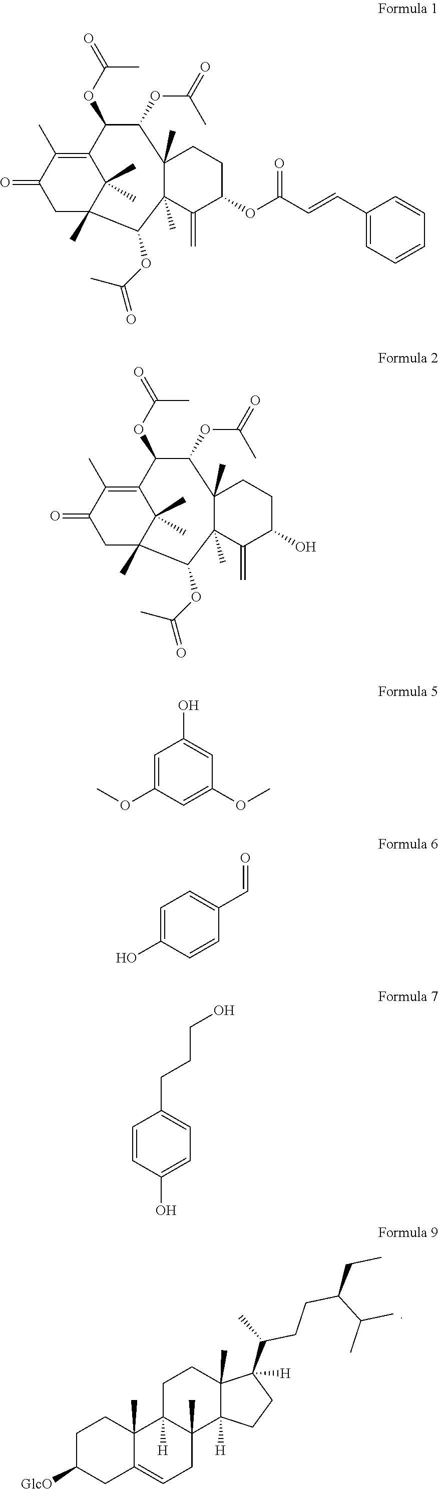

8. The pharmaceutical composition for preventing or treating allergic respiratory disease of claim 1, wherein Taxus cuspidata extract or fraction thereof further comprises one or more compounds selected from the group consisting of:
Sciadopitysin represented by the following formula (3),
Ginkgetin represented by the following formula (4), and/or
β-Sitosterol represented by the following formula (8);

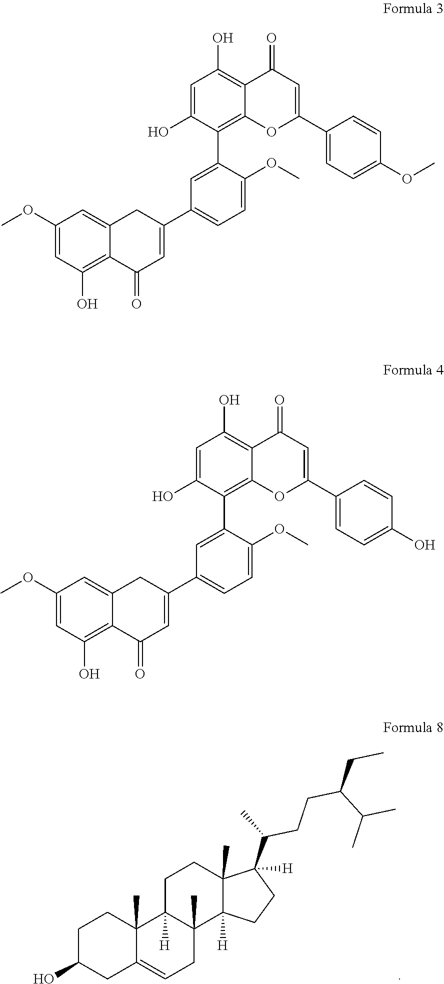

9. A health functional food composition for preventing or ameliorating allergic respiratory disease comprising Taxus cuspidata extract or a fraction thereof as an active ingredient.

10. The health functional food composition for preventing or ameliorating allergic respiratory disease of claim 9, wherein Taxus cuspidata extract is extracted using 40% (v/v) to 100% (v/v) ethanol as a solvent.

11. The health functional food composition for preventing or ameliorating allergic respiratory disease of claim 9, wherein the fraction is a methylene chloride (MC) fraction of a 40% (v/v) to 100% (v/v) ethanol extract of Taxus cuspidata.

12. The health functional food composition for preventing or ameliorating allergic respiratory disease of claim 9, wherein the allergic respiratory disease is allergic asthma, allergic bronchitis, or allergic rhinitis.

13. The health functional food composition for preventing or ameliorating allergic respiratory disease of claim 9, wherein Taxus cuspidata extract or fraction thereof inhibits the secretion of β-hexosaminidase, an allergen presents in the granules of mast cells, or inhibits the production of prostaglandin E2, TNF-α and interleukin-4 (IL-4) secreted by mast cells.

14. The health functional food composition for preventing or ameliorating allergic respiratory disease of claim 9, wherein Taxus cuspidata extract or fraction thereof inhibits or ameliorates allergic symptoms by reducing the number of eosinophils or inflammatory cells in the bronchi; reducing eosinophil infiltration; or reducing IgE in the serum.

15. The health functional food composition for preventing or ameliorating allergic respiratory disease of claim 9, wherein Taxus cuspidata extract or fraction thereof comprises one or more compounds selected from the group consisting of:
Taxinine represented by the following formula (1),
Taxinine A represented by the following formula (2),
Taxicatigenin represented by the following formula (5),
4-hydroxyl benzandehyde represented by the following formula (6),
3-(4-Hydroxyphenyl)-1-propanol (3-(4-Hydroxyphenyl)-1-propanol) represented by the following formula (7), and/or
Daucosterol represented by the following formula (9);

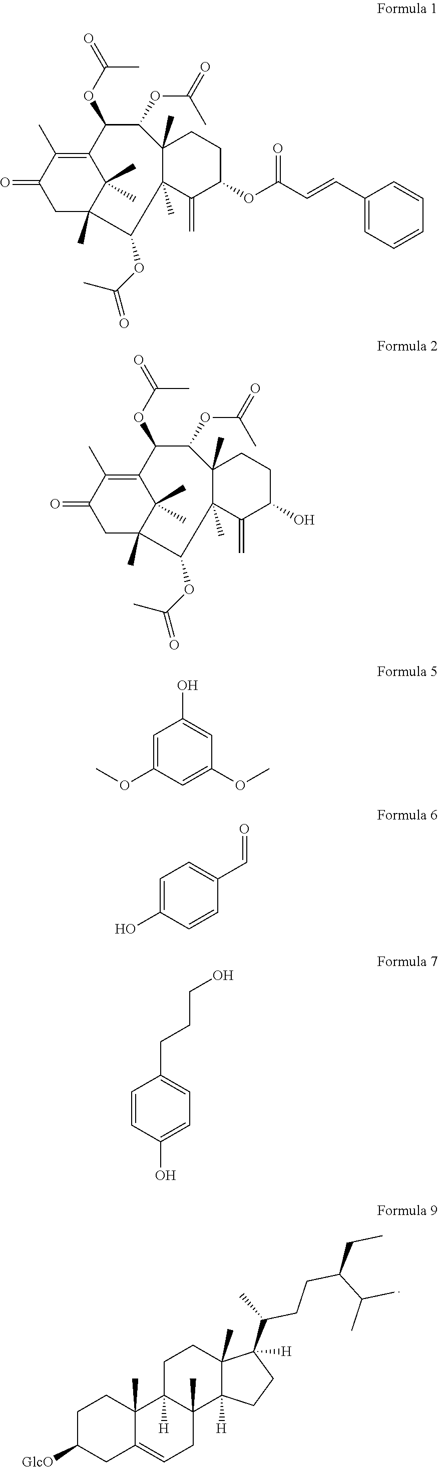

16. The health functional food composition for preventing or ameliorating allergic respiratory disease of claim 9, wherein Taxus cuspidata extract or fraction thereof further comprises one or more compounds selected from the group consisting of:
Sciadopitysin represented by the following formula (3),
Ginkgetin represented by the following formula (4), and/or
β-Sitosterol represented by the following formula (8);

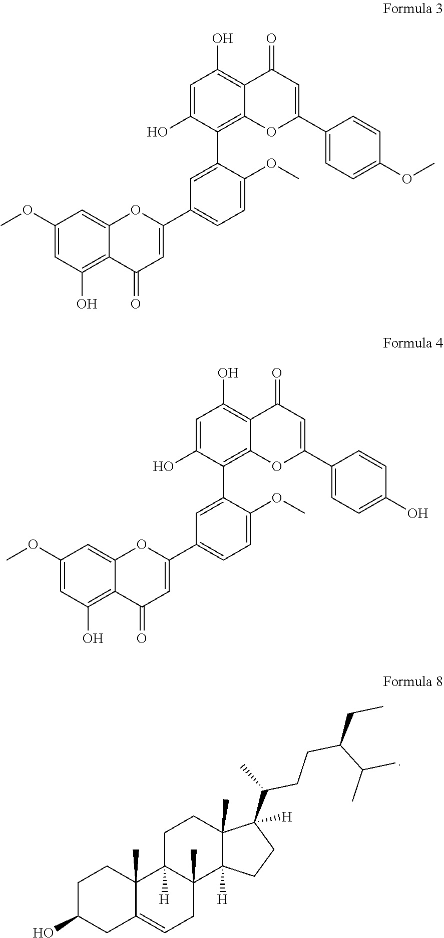

17. A method for preventing or treating allergic respiratory disease comprising administering or taking a composition comprising Taxus cuspidata extract or a fraction thereof as an active ingredient to an individual in need.
